(19) United States
(12) Patent Application Publication
(10) Pub. No.: US 2016/0120936 A1
(43) Pub. Date: May 5, 2016

(54) METHODS OF PREVENTING SURGICAL SITE INFECTIONS

(71) Applicant: Karen Troxel, Warsaw, IN (US)

(72) Inventor: Karen Troxel, Warsaw, IN (US)

(21) Appl. No.: 14/928,281

(22) Filed: Oct. 30, 2015

Related U.S. Application Data
(60) Provisional application No. 62/072,836, filed on Oct. 30, 2014.

(51) Int. Cl. A61K 38/16; B65D 81/32; B65D 25/48

(52) U.S. Cl. CPC A61K 38/16; B65D 25/48; B65D 81/32

(57) ABSTRACT

Methods for preventing a tissue infection associated with the site of a tissue disruption, such as a surgical incision. The methods include contacting tissue at the site with a composition comprising ε-polylysine in a physiologically-acceptable carrier, such as an isotonic solution, powder or hemostatic material containing ε-polylysine. Also provided are kits for preparing antibacterial ε-polylysine compositions.

### CLAIM OF PRIORITY

This application claims the benefit of U.S. Provisional Patent Application Ser. No. 62/072,836, filed on Oct. 30, 2014, the benefit of priority of which is claimed hereby, and which is incorporated by reference herein in its entirety.

### INTRODUCTION

The present technology relates to compositions for preventing infections associated with surgical procedures or other tissue disruptions.

Surgical site infections are infections that develop in patients after surgery at the part of the body where the surgery was performed. Whereas some surgical site infections are limited to the skin at a surgical site, other infections develop in underlying organs or other tissues, or on implanted devices or materials. Specifically, surgical site infections are caused by pathogens, such as bacteria, viruses, and fungi, that enter a patient's body through a surgical incision. The pathogen can come, for example, from the patient's own skin, mucous membranes, or gastrointestinal tract, from the attire of a medical professional, from lapses in aseptic techniques (such as hand hygiene), from ventilation, or from medical tools, equipment, or materials. About 2%-5% of patients undergoing inpatient surgery develop a surgical site infection. In the United States alone, about 300,000 patients are diagnosed with surgical site infections every year. Moreover, surgical site infections result in a mortality of about 3%.

Not only are surgical site infections a problem medically, they are also a problem economically. It costs anywhere from $3,000 to $29,000 to treat a surgical site infection, depending on the treatment procedure and the pathogen causing the infection. In the United States, about $10,000,000,000 is spent each year for the treatment of infections resulting from surgeries. Additionally, many pathogens, such as bacteria and fungi, are becoming increasingly resistant to commonly used antibiotics and antifungals, which results in increased costs and mortality associated with treating infections associated with surgery. Therefore, it is beneficial to prevent surgical site infections from developing at the onset.

Healthcare providers take measures to prevent infections resulting from a surgery. For example, healthcare providers practice proper hand hygiene (washing with soap and antiseptic agents), they wear protective garments, such as gowns, masks, and gloves, they may remove a patient's hair near the site of a forthcoming incision, they may administer antibiotics to a patient prior to surgery, and they clean the skin at surgical sites with antibacterial soaps. However, even when all precautionary measures are being followed, surgical site infections still occur.

Accordingly, health care providers have few, if any, clinically viable methods for reliably preventing surgical infections in cases where patient-related and procedure-related risk factors for surgical site infection have been identified. Thus, there remains a need to develop novel compositions and methods for use in surgical procedures or preventing infections incident to other tissue disruptions. There is a particular need for systems that are safe, easy to use, and afford sufficient flexibility for surgeons and other health care providers to determine where prophylactic antibacterial treatment is needed based upon patient risk factors and procedural risk factors.

### SUMMARY

The present technology provides methods for treating or preventing infections at the site of tissue disruption, such as a surgical site. Methods comprise administering an antibacterial composition comprising epsilon-polylysine (ε-polylysine) and a physiologically-acceptable carrier to the site of the tissue disruption. In various embodiments, administering can include lavaging, washing, or flushing tissues (e.g., skin, muscle or connective tissues) liquid compositions, or dusting powder compositions, at or proximate to the disruption. The disruption can be a result of a trauma that requires medical treatment or an incision made by a healthcare provider during a surgical procedure.

The present technology additionally provides kits for preparing antibacterial compositions for use in preventing infections at the site of a tissue disruption. The kit includes a first container containing ε-polylysine powder, a second container containing an isotonic solution. The kit may also contain a nozzle or other delivery mechanism operable to aid in the delivery of the composition to the site of the tissue disruption. For example, the nozzle may be a spray nozzle or a squirt nozzle, and may be configured for connection to at least one of the first container or the second container. The ε-polylysine and the isotonic solution can be combined in either the first container or the second container to generate an antibacterial composition with a predetermined concentration of ε-polylysine.

To better illustrate the orthopedic implant and related methods disclosed herein, a non-limiting list of examples is provided here:

In Example 1, a method for preventing a tissue infection at the site of a tissue disruption can comprise administering an antibacterial composition comprising ε-polylysine and a physiologically-acceptable carrier to the site of the disruption.

In Example 2, the method of Example 1 can optionally be configured such that the tissue disruption is an incision made during a surgical procedure.

In Example 3, the method of Example 2 can optionally be configured to further include implanting an orthopedic implant or other medical device.

In Example 4, the method of Example 1 can optionally be configured such that the tissue disruption is a result of a trauma.

In Example 5, the method of any one or any combination of Examples 1-3 can optionally be configured such that the tissue is skin, muscle, or connective tissue.

In Example 6, the method of any one or any combination of Examples 1-5 can optionally be configured such that the antibacterial composition comprises from about 200 to about 5000 μg of the ε-polylysine per ml of the composition.

In Example 7, the method of any one or any combination of Examples 1-6 can optionally be configured such that the composition is a liquid.

In Example 8, the method of Example 7 can optionally be configured such that the carrier comprises is an isotonic solution selected from the group consisting of saline, phosphate buffered saline, lactated Ringer's solution, Ringer's solution, 5% dextrose in water, and combinations thereof.

In Example 9, the method of Example 7 can optionally be configured such that the carrier comprises a thickener selected from the group consisting of acacia, mucilage, alginic acid, sodium alginate, tragacanth, bentonite, starch, carbomers, poloxamers, gelatin, xanthan gum, polyvinyl alcohol, magnesium aluminum silicate, methylcellulose, carboxymethylcellulose, croscarmellose sodium, ethylcellulose, hydroxyethylcellulose, hydroxypropylcellulose, hydroxypropylmethylcellulose, and combinations thereof.

In Example 10, the method of Example 7 can optionally be configured such that the antibacterial composition is obtained from a sealed and sterilized kit comprising a first container containing the ε-polylysine and a second container containing an isotonic solution or other liquid carrier.

In Example 11, the method of any one or any combination of Examples 7-10 can optionally be configured to further include applying the composition to tissue adjacent to the tissue disruption.

In Example 12, the method of any one or any combination of Examples 1-6 can optionally be configured such that the composition is a non-liquid.

In Example 13, the method of Example 12 can optionally be configured such that the composition is a powder.

In Example 14, the method of Example 13 can optionally be configured such that the carrier comprises a solid filler, diluent, or glidant.

In Example 15, the method of Example 14 can optionally be configured such that the carrier comprises a solid an excipient selected from the group consisting of starch, talc, magnesium stearate, lactose, collagen, starch, and combinations thereof.

In Example 16, the method of any one or any combination of Examples 12-15 can optionally be configured such that the antibacterial composition comprises a hemostatic material.

In Example 17, the method of Example 16 can optionally be configured such that the hemostatic material comprises collagen or starch.

In Example 18, the method of Example 12 can optionally be configured such that the administering comprises dusting the tissue with the composition.

In Example 19, the method of any one or any combination of Examples 1-18 can optionally be configured such that the administering comprises contacting the site with a hemostatic material comprising the antibacterial composition.

In Example 20, the method of Example 19 can optionally be configured such that the hemostatic material is a sponge, foam, cloth, gauze, or powder.

In Example 21, the method of Example 12 can optionally be configured such that the carrier is a bone cement.

In Example 22, the method of Example 21 can optionally be configured such that the bone cement is a polymethyl methacrylate (PMMA) cement, preferably comprising from about 2% to about 20% ε-polylysine.

In Example 23, a system for preparing an antibacterial composition for use in preventing a tissue infection at the site of a tissue disruption can comprise a first container containing ε-polylysine powder and a second container containing an isotonic solution.

In Example 24, the system of Example 23 can optionally be configured to further include a nozzle configured for attachment to at least one of the first container and the second container, such as by threading.

In Example 25, the system of Example 24 can optionally be configured such that the nozzle is a spray nozzle or a squirt nozzle.

In Example 26, the system of any one or any combination of Examples 23-25 can optionally be configured such that the system is a kit that is sealed and sterilized.

In Example 27, the system of any one or any combination of Examples 23-26 can optionally be configured such that the first container contains a predetermined amount of ε-polylysine powder and the second container contains a predetermined amount of the isotonic solution, such that when all of the isotonic solution is transferred to the first container, the antibacterial composition is formed with a predetermined concentration of ε-polylysine.

In Example 28, the system of Example 27 can optionally be configured such that the predetermined concentration of the ε-polylysine is from about 200 to about 5000 μg/mL.

In Example 29, the system of any one or any combination of Examples 23-28 can optionally be configured such that the isotonic solution is selected from the group consisting of saline, phosphate buffered saline, lactated Ringer's solution, Ringer's solution, 5% dextrose in water, and combinations thereof.

In Example 30, the method or system of any one or any combination of Examples 1-29 is optionally configured such that all elements or options recited are available to use or select from.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. A non-limiting discussion of terms and phrases intended to aid understanding of the present technology is provided at the end of this Detailed Description.

The present technology provides compositions, methods, kits and systems for treating or preventing infections associated with tissue disruptions. As further discussed herein, such compositions, methods, kits and systems use epsilon polylysine, preferably in a composition comprising a physiologically-acceptable carrier.

Epsilon polylysine (ε-polylysine) is a naturally produced polymer of the amino acid, lysine. Epsilon (ε) refers to the linkage of the lysine molecules. In contrast to a normal peptide bond wherein a backbone carboxyl group of a first amino acid forms a bond with a backbone amino group of a second amino acid, the peptide bond in ε-polylysine is formed between a backbone carboxyl group of a first lysine molecule and a side chain ε-amino group of a second lysine molecule. Commercially, ε-polylysine, with chains of about 25 to about 30 lysine molecules is used as a preservative in food and cosmetics. In various embodiments, the ε-polylysine has a chain length of about 10 or more lysine residues. For example, the ε-polylysine may have a chain length of from about 10 to about 40 lysine residues, from about 20 to about 35 lysine residues, or from about 25 to about 30 lysine residues.

Epsilon polylysine may be produced in a variety of methods, including methods among those known in the art, such as by isolation from natural sources (e.g., microbial production) or synthetic product. For example, ε-polylysine can be produced by fermentation of Streptomyces albulus.

Without limiting the scope, function or mechanism of the present technology, it is believed that ε-polylysine destabilizes cell membranes in Gram-negative bacteria and Gram-positive bacteria. A non-limiting advantage of ε-polylysine's mechanism for antibacterial activity in the present technology is that it is highly improbable for microorganisms to develop resistance to it. Moreover, ε-polylysine is nontoxic to mammalian cells at concentrations far in excess of its antibacterially-effective concentrations. The recommended dosage in food processing is 25 to 125 ppm, which is equivalent to 25 to 125 μg/mL when the product is a liquid.

### Compositions

As discussed above, the present technology provides compositions comprising ε-polylysine in a physiologically-acceptable carrier. As used herein, a “physiologically-acceptable” component or carrier is one that is suitable for use with humans and/or animals without undue adverse side effects (such as toxicity, irritation, and allergic response) commensurate with a reasonable benefit/risk ratio. The compositions preferably comprise a safe and effective amount of ε-polylysine. A “safe and effective” amount of ε-polylysine is an amount that is sufficient to have the desired infection inhibitory effect in the human or lower animal subject, without undue adverse side effects (such as toxicity, irritation, or allergic response), commensurate with a reasonable benefit/risk ratio when used in the manner of this technology. The specific safe and effective amount of ε-polylysine will, obviously, vary with such factors as the particular tissue disruption being treated (e.g., the specific surgical procedure), the physical condition of the patient, the condition and characteristics of the tissue to be treated, the nature of concurrent therapy (if any), the specific route of administration, and the specific composition of the carrier and other components in the composition. As further discussed below, the safe and effective amount is preferably has an infection-inhibiting effect that substantially reduces the number of microbes that contact and grow on tissues exposed at the tissue disruption relative to the number of microbes that would be present on the tissue without administration of the antibacterial composition.

In various embodiments, the compositions of the present technology are liquid compositions comprising a safe and effective amount of ε-polylysine in a liquid carrier. Suitable carriers include water and other components that, with ε-polylysine, form an isotonic solution. As discussed below, such solutions are operable to deliver the ε-polylysine to a site of tissue disruption when sprayed, squirted or rubbed (such as with a sponge, foam, cloth, or gauze) on the tissue.

In various embodiments, the antibacterial composition comprises from about 200 μg to about 5000 μg ε-polylysine per mL. For example, the antibacterial composition may comprises ε-polylysine at a concentration of about 200 μg/mL, about 500 μg/mL, about 750 μg/mL, about 1000 μg/mL, about 1250 μg/mL, about 1500 μg/mL, about 1750 μg/mL, about 2000 μg/mL, about 2250 μg/mL, about 2500 μg/mL, about 2750 μg/mL, about 3000 μg/mL, about 3250 μg/mL, about 3500 μg/mL, about 3750 μg/mL, about 4000 μg/mL, about 4250 μg/mL, about 4500 μg/mL, about 4750 μg/mL, or about 5000 μg/mL.

The isotonic solution can be any non-toxic isotonic solution commonly used in the art. Non-limiting examples of isotonic solutions include saline, phosphate-buffered saline, Ringer's Solution, lactated Ringer's solution, and dextrose in water. A typical saline solution includes about 0.9% NaCl in water. Ringer's solution typically includes about 147 mEq sodium, about 4 mEq potassium, about 4 mEq calcium, and about 155 mEq chloride in water. Lactated Ringer's solution typically includes about 130 mEq sodium, about 4 mEq potassium, about 3 mEq calcium, about 109 mEq chloride, about 28 mEq sodium, and lactate and water. Dextrose in water can be about 5% dextrose in water (D5W). Nonetheless, the concentrations of the components of the foregoing solutions can be varied so long as the solutions remain isotonic.

In various embodiments, the antibacterial composition consists of ε-polylysine and saline, phosphate buffered saline, or other isotonic solution, wherein the ε-polylysine has a concentration of from about 200 μg/mL to about 5000 μg/mL. In other embodiments, the antibacterial composition comprises ε-polylysine and saline, phosphate buffered saline, or other isotonic solution, wherein the ε-polylysine has a concentration of from about 200 μg/mL to about 5000 μg/mL.

In various embodiments, the antibacterial compositions consist of, or consist essentially of ε-polylysine and a physiologically acceptable carrier. In such embodiments, the carrier consists of or consists essentially of a solvent for ε-polylysine and, optionally, an additive material. Additive materials useful in the compositions and methods of this technology include antioxidants, colorants, viscosity modifying agents, and therapeutic actives. Non-limiting examples of viscosity modifying agents include acacia, mucilage, alginic acid, sodium alginate, tragacanth, bentonite, starch, carbomers, poloxamers, gelatin, xanthan gum, polyvinyl alcohol, magnesium aluminum silicate, methylcellulose, carboxymethylcellulose, croscarmellose sodium, ethylcellulose, hydroxyethylcellulose, hydroxypropylcellulose, hydroxypropylmethylcellulose, and combinations thereof. In various embodiments, the compositions do not contain additional antibacterial agents.

In some embodiments, the additive component is one or more active agent in addition to the ε-polylysine. The active agent can be an antibacterial, antibiotic, antioxidant, anesthetic, (such as bupivacaine), small molecule drug, or anti-inflammatory solution. Antibiotics useful herein include, for example, rifamycins (such as rifampin), fosfomycin, fusidic acid, glycylcyclines, aminoglycosides, quinolones, glycopeptides, bismuth thiols, sulfonamides, trimethoprim, macrolides, oxazolidinones, β-lactams, lincosamides, chloramphenicol, gramicidins, polymyxins, lipodepsipeptides, bacitracins, tetracyclines (such as minocycline), penicillin, ampicillin, cefazolin, clindamycin, erythromycins, levofloxacin, vancomycin, and mixtures thereof

Tetracycline antibiotics refer to a number of antibiotics of either natural, or semi-synthetic origin, derived from a system of four linearly annealed six-membered rings (1,4,4a,5,5a,6,11,12a-octahydronaphthacene) with a characteristic arrangement of double bonds. The tetracycline antibiotic can include one or more tetracyclines, and/or semi-synthetic tetracyclines such as doxycycline, oxytetracycline, demeclocycline, lymecycline, chlortetracycline, tigecycline and minocycline. A preferred tetracycline is minocycline or minocycline hydrochloride. The amount of tetracycline present in the infection-inhibiting coating can range from about 5 μg/cm² to about 1000 μg/cm², or from about 10 μg/cm² to about 800 μg/cm².

Rifamycin class of antibiotics is a subclass of antibiotics from the ansamycin family of antibiotics. The present antibiotic agent or agents can include one or more rifamycin antibiotics from the group rifamycin B, rifampin or rifampicin, rifabutin, rifapentine and rifaximin. Rifampin is commercially available as Rifadin and Rimactane from Sanofi-Aventis U.S. LLC. (Bridgewater, N.J., USA).

Antibacterial peptides useful herein include, for example, host defense proteins, defensins, magainins, cathetlicidins, protegrins, antibiotics, nisins, and synthetic mimics of host defense proteins such as cationic steroids. Antiseptics and disinfectants include, for example, chlorhexidine, polyhexanide, triclosan, and iodine-delivering formulas such as betadine or povidone-iodine. Metal ions include various formulations of silver that effectively release silver ions, including silver salts and silver nanoparticles, or copper salts and copper nanoparticles that release copper ions.

Other antibacterial agents useful herein include salicylic acid and its metabolite methyl salicylate, and sugar alcohols and polyols (such as xylitol and erythritol). Such sugar alcohols can have antibacterial properties by preventing bacterial adhesion or bacterial biofilm formation. Polysaccharides, such as chitosan and alginate, are also useful herein. Additionally, the active agent can include bisphosphonates, insulin mimetics (such as vanadium compounds, including vanadyl acetylacetonate), growth factors, and cytokines

The liquid compositions according to the present technology can be sterilized using appropriate sterilization methods among those known in the art.

### Non-Liquid Compositions

The present technology provides non-liquid compositions, which may be solids (e.g., powders) or non-flowable semi-solids (e.g., pastes). Such compositions generally comprise ε-polylysine and a physiologically-acceptable carrier. The carriers may be aqueous or non-aqueous. It is understood, though, that in some embodiments methods of the present technology may consist of applying ε-polylysine in powder form to the site of a tissue disruption. Thus, in such embodiments, the composition consists of ε-polylysine. In other embodiments, the antibacterial comprises ε-polylysine and a solid or semi-solid carrier. The carrier may comprise one or more fillers or glidants, such as selected from the group consisting of starch, talc, magnesium stearate, lactose, collagen, starch, and combinations thereof.

Compositions may also comprise one or more additive materials including additive materials described above regarding liquid compositions. For example, the antibacterial composition may comprise one or more additive components, such as antioxidants, colorants, viscosity modifying agents, and therapeutic actives. Such additive materials may also include lipids, such as fatty acids, triacylglycerols, diacylgycerols, glycerophospholipids (such as synthetic and naturally-derived phosphatidylcholine), and mixtures thereof. Such compositions, dispensing devices and methods are disclosed in U.S. Patent Application Publication 2013/0288951, Troxel et al., published Oct. 31, 2013, incorporated by reference herein.

The compositions may comprise a hemostatic material, for controlling or inhibiting bleeding. Such materials include starch, collagen, bone wax, acrylates, aluminum chloride, silver nitrate, zeolites, kaolin, thrombin, and lipid materials. Hemostatic agents among those useful herein include phospholipids, as described in U.S. Patent Application Publication 2014/0274875, Troxel et al., published Sep. 18, 2014, incorporated by reference herein.

In various embodiments, the solid composition comprises a ε-polylysine in a bone cement carrier. The bone cement can be any bone cement known in the art. For example, such bone cement may be a methacrylate cement, such as those comprising polymethyl methacrylate (PMMA) and an activation agent which induces polymerization to generate the bone cement. The bone cement may contain ε-polylysine at a concentration of about 2% to about 10% by weight and can be sterilized prior to hardening. As a non-limiting example, the bone cement can be Cobalt™ MV Bone Cement bone cement from Biomet Inc. (Warsaw, Ind.). Cobalt™ MV Bone Cement comprises a powder component, including methyl methacrylate-styrene copolymer, PMMA, zirconium dioxide, blue pigment, and benzoyl peroxide (catalyst), and a liquid component comprising methylmethacrylate and N, N-dimethyl-p-toluidine. When the powder and liquid components are mixed, the N, N-dimethyl-p-toluidine activates the benzoyl peroxide catalyst, which initiates polymerization. Epsilon polylysine powder can be added to the bone cement powder component prior to adding the liquid component to obtain an antibacterial bone cement comprising ε-polylysine. The ε-polylysine inhibits microbial growth on the surface of the bone cement after it has hardened, for example, in a subject after an orthopedic procedure. Therefore, bone cement comprising ε-polylysine can be used for local prevention of a tissue infection associated with bacteria at a site of a medical device implanted in a subject with bone cement. The bone cement can be used as an adhesive for medical implants. Antibacterial bone cements can be made and then sterilized, or they can be made with sterile components under sterile conditions.

In various embodiments, the antibacterial composition comprises ε-polylysine and a hemostatic absorbable powder. The ε-polylysine may be incorporated in the composition at a concentration from about 2% to about 35% by weight of the antibacterial composition. In some embodiments, the hemostatic material is an absorbable powder comprising collagen, polysaccharide or starch. The material comprising ε-polylysine is a sponge, foam, cloth, gauze, or powder.

The present technology provides methods for treating or preventing infections at a site of a tissue disruption. Compositions and methods of the present technology are operable to prevent, inhibit, microbial infections or growth in human or non-human subjects. As used herein, “infections” refers to the presence, growth or colonization of microbes and microorganisms, such as bacteria, yeast, or other fungal organisms, in a subject.

Without limiting the scope, function or utility of the present technology, it is understood that methods of preventing are effected by inhibiting microbial growth by contacting tissue in which microbes are, or may be, present, with ε-polylysine. In some embodiments, microbial growth is substantially prevented or suppressed, so as to allow the immune system of the treated subject to recognize and neutralize remaining microbes. In some embodiments, the subject having a tissue disruption may be at risk of infection, due to microbes that are present and not detected or microbes that may be later introduced to the site of the disruption. Thus, the methods of prevention reduce the likelihood of infection at the site of the tissue disruption in such subjects. Indeed, as to any subject not having an infection at the time of administration of ε-polylysine, it may not be definitively known whether the subsequent lack of infection was, in fact, due to the administration of ε-polylysine or for some other reasons (i.e., an actual lack of microbial exposure). Rather, the effectiveness of the method of preventing infection may be seen only in the reduction of the incidence of infections in a population of subjects or tissue disruptions at risk of infection. Thus, as used herein, “preventing” refers to reducing the rate or probability of infections associated with a tissue disruption, in a subject at risk of infection.

In surgical procedures, microbes (“target microbes”) include organisms that are associated with surgical tools or non-surgical objects that pierce a subject's skin, which may contact bone or surrounding tissues such as skin, blood, muscle, or cartilage. Thus, any microbe that has the potential to enter surreptitiously and colonize at a surgical site or area of trauma may be targeted in accordance with the present technology. Target microbes of particular concern are those that colonize the skin of a surgical subject, since these organisms may enter the subject at the site of a wound.

Particularly relevant target microbes include Gram-positive and Gram-negative bacteria. Such organisms include Klebsiella, Enterobacter, Acinetobacter, Pseudomonas, Escherichia, and Staphylococcus. Specific bacteria include Staphylococcus aureus, as represented by strain NCTC 8325 and methicillin resistant strains which presently cause significant problems in hospital environments. Further targets are Staphylococcus epidermidis, represented by strain NCTC 11047. Some of these bacteria are known to produce fibrinogen-binding clumping factors A and B and the fibronectin-binding protein (FnbA), capable of adhering to orthopedic implants and related devices.

The methods of the present technology comprise administering ε-polylysine or composition containing ε-polylysine to the site of a tissue disruption. The disruption can be, for example, the result of a wound, such as from an accident or other trauma, a surgical incision or other disruption of tissue (including laparoscopic and endoscopic procedures) made by a surgeon or other medical professional, or a disruption caused by a tissue disorder such as a diabetic skin ulcer. For minor wounds, such as cuts or scrapes, the treatment can be administered by a non-medical professional. However, more serious wounds, such as those requiring surgery and or sutures, or wounds resulting from a surgical incision, the medical treatment can be administered by a medical professional or healthcare provider, such as, for example, a physician, physician's assistant, nurse, or other medical technician associated with a microorganism at a site of a tissue disruption. Surgical procedures may be associated with colon surgery, appendectomy, heart surgery, surgeries in the pelvis such as hysterectomy, or after a C-section birth to prevent infection of the C-section surgical wound.

Administering comprises administering a safe and effective amount of ε-polylysine to the site of a tissue disruption. In various embodiments, administration of ε-polylysine may be to the disrupted tissue and to surrounding tissues.

For liquid compositions, such as those described above, administering include lavaging, washing, or flushing the tissues with the antibacterial composition. Lavaging, washing, or flushing internal tissues with compositions according to the present technology reduces the probability of a surgery-associated infection at or near the site of a surgical wound or incision. In some embodiments, lavaging, washing, or flushing is performed any time during a surgical procedure, including at a time immediately proximate to closing the surgical wound. Lavaging, washing, or flushing can be performed by spraying or squirting the antibacterial composition onto tissues exposed by the wound. In some embodiments, methods further comprise covering the wound with a bandage or other suitable dressing after administering or contacting.

In some embodiments, methods comprise contacting a powder containing ε-polylysine to the site of the tissue disruption. Administering can be performed by manually dusting the at least one tissue with the powder or by shaking a container containing the powder over the at least one tissue, wherein the container comprising a surface with plurality of small holes (similar to a salt shaker). In one embodiment, administering is performed with a device that blows the powder in a desired direction when activated.

In various embodiments, methods may comprise use of a hemostatic material or other composition or device to effect delivery of the ε-polylysine or composition comprising ε-polylysine to the site of tissue disruption. Such a device may be a hemostatic material, such as sponge, foam, cloth, gauze or powder mixed or infused with an antibacterial composition comprising ε-polylysine as discussed above. Contacting is performed by dabbing or rubbing, spraying or otherwise applying the material to the disrupted tissue. Optionally, the material is applied to tissue (e.g., skin) adjacent to and/or surrounding the site of the tissue disruption.

### Kits

The present technology provides devices and kits for preparing and, optionally, administering antibacterial compositions comprising ε-polylysine. For example, a kit may comprise a first container containing ε-polylysine powder, and a second container containing a solvent, such as an isotonic solution, suitable for dissolving the ε-polylysine. The first or second containers can be small sealed bag, a sealed packaging (such as aluminum), or a bottle. For example, in some embodiments, the first container is a bag or sealed packaging that can be manually opened so that all of the ε-polylysine powder contained therein can be easily transferred to the second container. In other embodiments, the first container is a bottle. The bottle can be sealed by a cap, lid, or the nozzle. Therefore, the cap, lid or nozzle can be removed, all of the contents of the second container can be transferred to the first container, and the first container can be covered with the nozzle.

The first container contains a predetermined amount of ε-polylysine powder and the second container contains a predetermined amount of the isotonic solution, as described above. When the contents of the first and second containers are combined, the antibacterial composition is formed with a predetermined concentration of ε-polylysine. In various embodiments, the predetermined concentration of the ε-polylysine is from about 200 to about 5000 μg/mL. After combining, the ε-polylysine and the isotonic solution may be mixed, such as by shaking or any other suitable method to effect dissolution of the ε-polylysine. Mixing is complete when all of the ε-polylysine is dissolved in the isotonic solution to generate the antibacterial composition.

The kit preferably comprises sealed packaging that is airtight with a barrier to water, with a sterile inner packaging barrier. The kit can be sterilized by conventional means, including gamma radiation or electron beam radiation without a loss of antibacterial activity.

In some embodiments, the kit may comprise a nozzle or other device operable to effect delivery of the antibacterial composition to disrupted tissue in a method of this technology. For example, the device may be a spray nozzle, a squirt nozzle, or any other nozzle used in the art. The nozzle can be permanently affixed or removably coupled to at least one of the first container or the second container, for example, by threading, luer fittings, bayonet slots, interference fit, or by any other means generally available in the art. In some embodiments, the kit comprises a removable nozzle which is not attached to either container, but is configured to be attached to either the first or second container in a method of this technology after the ε-polylysine and isotonic solution are mixed. Preferably, when the nozzle is coupled to either the first container or the second container, the antibacterial solution cannot leak. In other words, the antibacterial composition is only expelled from the container by activating the nozzle. When the nozzle is a spray nozzle, the antibacterial composition can be sprayed from the container by pressing on a trigger, such as in a spray bottle. When the nozzle is a squirt nozzle, the antibacterial composition can be squirted from the container by manually squeezing the container.

In one embodiment, the antibacterial composition is prepared from the kit by transferring the ε-polylysine to the second container, coupling the nozzle to the second container, and mixing the ε-polylysine with the isotonic solution. In another embodiment, the antibacterial composition is prepared by transferring the isotonic solution to the first container, coupling the nozzle to the first container, and mixing the ε-polylysine with the isotonic solution. Mixing can be performed by any means known in the art, such as, for example, by inverting the container or by manually rotating the container to generate a swirling action within the container. Mixing is complete when all of the ε-polylysine is dissolved in the isotonic solution to generate the antibacterial composition. After the antibacterial composition has been made, the antibacterial composition can be sterilized, for example, by radiation.

In some embodiments, the method comprises obtaining a sealed and sterilized kit comprising a first container containing the ε-polylysine, a second container containing the isotonic solution, and a nozzle, wherein the nozzle is one of a spray nozzle or a squirt nozzle. In such embodiments, the method also comprises transferring the isotonic solution from the second container into the first container and coupling the nozzle to the first container or transferring the ε-polylysine from the first container to the second container and coupling the nozzle to the second container. After the antibacterial composition is made, the antibacterial composition can be sterilized, for example, by radiation.

### Non-Limiting Discussion of Terminology

The headings (such as “Introduction” and “Summary”) and sub-headings used herein are intended only for general organization of topics within the present technology, and are not intended to limit the disclosure of the present technology or any aspect thereof. In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The citation of references herein does not constitute an admission that those references are prior art or have any relevance to the patentability of the technology disclosed herein. Any discussion of the content of references cited in the Introduction is intended merely to provide a general summary of assertions made by the authors of the references, and does not constitute an admission as to the accuracy of the content of such references. All references cited in the “Description” section of this specification are hereby incorporated by reference in their entirety.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

As used herein, the term “operable” refers to a material, device or action which is capable, by virtue of its composition, design or features, to perform a recited function. In some embodiments, an operable material device or action is adapted to perform the function, having a specific composition, design or feature that is adapted (relative to similar composition, design or features known in the art), individually or in combination with other composition, design and features of the present technology, for use in performing the recited function. An operable material, device or action may, in some embodiments, also be capable of performing other functions.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of” Thus, for any given embodiment reciting ingredients, components or process steps, Applicants specifically envision embodiments consisting of, or consisting essentially of, such ingredients, components or processes excluding additional ingredients, components or processes (for consisting of) and excluding additional ingredients, components or processes affecting the novel properties of the embodiment (for consisting essentially of), even though such additional ingredients, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

What is claimed is:

1. A method for preventing a tissue infection at the site of a tissue disruption, the method comprising: administering an antibacterial composition comprising ε-polylysine and a physiologically-acceptable carrier to the site of the disruption.

2. The method according to claim 1, wherein the tissue disruption is an incision made during a surgical procedure.

3. The method according to claim 2, wherein the surgical procedure comprises implanting an orthopedic implant or other medical device.

4. The method according to claim 1, wherein the tissue is skin, muscle, or connective tissue.

5. The method according to claim 1, wherein the antibacterial composition comprises from about 200 to about 5000 μg of the ε-polylysine per ml of the composition.

6. The method according to claim 1, wherein the antibacterial composition is a liquid.

7. The method according to claim 6, wherein the carrier comprises an isotonic solution selected from the group consisting of saline, phosphate buffered saline, lactated Ringer's solution, Ringer's solution, 5% dextrose in water, and combinations thereof.

8. The method according to claim 6, wherein the carrier comprises a thickener selected from the group consisting of acacia, mucilage, alginic acid, sodium alginate, tragacanth, bentonite, starch, carbomers, poloxamers, gelatin, xanthan gum, polyvinyl alcohol, magnesium aluminum silicate, methylcellulose, carboxymethylcellulose, croscarmellose sodium, ethylcellulose, hydroxyethylcellulose, hydroxypropylcellulose, hydroxypropylmethylcellulose, and combinations thereof.

9. The method according to claim 6, wherein the antibacterial composition is obtained from a sealed and sterilized kit comprising a first container containing the ε-polylysine and a second container containing an isotonic solution or other liquid carrier.

10. The method according to claim 6, wherein the method comprises applying the antibacterial composition to tissue adjacent to the tissue disruption.

11. The method according to claim 1, wherein the antibacterial composition is a powder.

12. The method according to claim 11, wherein the carrier comprises a solid filler, diluent, or glidant.

13. The method according to claim 11, wherein the administering comprises dusting the tissue with the antibacterial composition.

14. A system for preparing an antibacterial composition for use in preventing a tissue infection at the site of a tissue disruption, the system comprising:
a first container containing ε-polylysine powder; and
a second container containing an isotonic solution.

15. The system according to claim 14, further comprising a nozzle configured for attachment to at least one of the first container and the second container.

16. The system according to claim 15, wherein the nozzle is a spray nozzle or a squirt nozzle.

17. The system according to claim 14, wherein the system is a kit that is sealed and sterilized.

18. The system according to claim 14, wherein the first container contains a predetermined amount of ε-polylysine powder and the second container contains a predetermined amount of the isotonic solution, such that when all of the isotonic solution is transferred to the first container, the antibacterial composition is formed with a predetermined concentration of ε-polylysine.

19. The system according to claim 18, wherein the predetermined concentration of the ε-polylysine is from about 200 to about 5000 μg/mL.

20. The system according to claim 14, wherein the isotonic solution is selected from the group consisting of saline, phosphate buffered saline, lactated Ringer's solution, Ringer's solution, 5% dextrose in water, and combinations thereof.
